(19) United States
(12) Patent Application Publication
(10) Pub. No.: US 2024/0399135 A1
(43) Pub. Date: Dec. 5, 2024

(54) AXIAL-FLOW PUMP FOR A VENTRICULAR ASSIST DEVICE AND METHOD FOR PRODUCING AN AXIAL-FLOW PUMP FOR A VENTRICULAR ASSIST DEVICE

(71) Applicant: KARDION GMBH, Stuttgart (DE)

(72) Inventors: Ingo Stotz, Ditzingen (DE); Niko Baeuerle, Bad Liebenzell (DE)

(21) Appl. No.: 18/799,771

(22) Filed: Aug. 9, 2024

Related U.S. Application Data
(63) Continuation of application No. 17/057,252, filed on Jun. 18, 2021, now Pat. No. 12,064,615.

(30) Foreign Application Priority Data
May 30, 2018 (DE) 102018208541.2

(51) Int. Cl. A61M 60/237; A61M 60/13; A61M 60/178; A61M 60/806; F04D 3/02

(52) U.S. Cl. CPC A61M 60/237; A61M 60/13; A61M 60/178; A61M 60/806; F04D 3/02

(57) ABSTRACT

The disclosure relates to a cardiac support system for insertion through a catheter into a blood vessel, including an axial flow pump having a tubular housing, an intake portion disposed in a distal portion of the tubular housing, at least one peripheral outlet opening disposed in a proximal portion of the tubular housing, and a pump rotor disposed within the tubular housing, the pump rotor including a hub and at least one blade element wound at least partially helically around the hub and configured to draw blood into the intake portion toward the proximal portion of the tubular housing, where a portion of the at least one blade element is disposed proximal to a distal edge of the outlet opening, and the at least one blade element transitions from having a convex curvature to having a concave curvature in a region of the outlet opening.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The invention relates to an axial flow pump for a cardiac support system and a method for producing an axial flow pump for a cardiac support system. The invention further relates to a device for producing an axial flow pump for a cardiac support system and a computer program for executing and/or controlling such a production method.

### Description of the Related Art

To provide cardiovascular support for patients having heart failure, among other things, systems are used that take over part or all of the heart's pumping function. These systems, which are also referred to as cardiac support systems or VADs (ventricular assist devices) for short, can be subdivided into temporary systems for short-term heart support and permanent systems for long-term use on or in the patient. One component of such a system is usually a blood pump, typically a centrifugal pump (turbo pump), which is driven by an integrated electric motor and produces the required blood flow by means of an impeller. The pump can be implanted in different locations. The pump can be sutured to the heart from the outside by means of an invasive sternotomy, for example, or it can be placed into the aorta or into a ventricle in a minimally invasive manner by means of a catheter. In the latter case, the maximum permissible outer diameter of the pump is generally limited to 10 mm, which is why the use of an axial pump having an axial flow impeller is desirable. In the process, the blood to be conveyed is expelled through the outlet openings disposed on the circumference of a cylindrical pump housing in order to be returned to the aorta.

In a centrifugal pump, the rotation of an impeller introduces swirl into the flow. The swirl can be reduced, for example, by a downstream stationary guide wheel, also known as a stator.

### SUMMARY

The object of the invention is to provide an axial flow pump for a cardiac support system that can convey blood both efficiently and gently. This object is achieved by an axial flow pump and production method specified in the claims. Advantageous embodiments of the invention are specified in the dependent claims.

Such an axial flow pump for a cardiac support system, a method for producing such an axial flow pump for a cardiac support system, a device using said method, and a computer program for executing and/or controlling such a production method are described in the following.

The invention is based on the knowledge that particularly gentle and efficient fluid conveyance can be achieved by using a blade on an impeller for an axial centrifugal pump of a cardiac support system which is curved at least in sections in a wavy manner, in conjunction with a suitable positioning of the impeller relative to outlet openings on a pump housing.

The design of the impeller results in increased efficiency with reduced blood damage. Under certain boundary conditions, for example limited installation space, it makes sense to do without a fixed guide wheel, which usually results in a corresponding reduction in the efficiency of the pump. Using the design of the impeller or its blades and positioning them appropriately relative to the pump housing, it is now possible to at least partially compensate for the reduction in efficiency. This can also reduce blood damage caused by hemolysis.

An axial flow pump according to the invention can in particular have the following features:
a pump housing for arrangement in a blood vessel; and
a pump rotor which is or can be mounted in the pump housing such that it can rotate about an axis of rotation and consists of a hub and at least one blade element which is wound at least in sections helically around the hub for conveying a conveying medium in the direction of the axis of rotation, wherein the blade element comprises at least one blade section having a wavy blade curvature.

An axial flow pump can be understood to be a centrifugal pump having an integrated electric motor for axially drawing in a conveying medium. The drawn-in conveying medium can be expelled, for example in radial or diagonal direction, through one or more lateral outlet openings in the pump housing. A cardiac support system can in particular be understood to be a tube or hose-shaped pump device that can be placed into the aorta or into a ventricle in a minimally invasive manner by means of a catheter. The cardiac support system can have a maximum outer diameter between 10 and 15 mm, for example. A pump housing can be understood to be a housing in the form of a hose or a tube. A blood vessel can be understood to be a (main) artery or a ventricle. Accordingly, blood can be understood to be a conveying medium. A pump rotor can be understood to be an impeller of the axial flow pump into which fluid flows axially. The pump rotor can comprise, for example two or more blade elements, which can be wound at least in sections helically around the hub and can comprise at least one screw section having a wavy blade curvature. A wavy blade curvature can be understood to be an undulation of a camber line of the blade element, which is characterized by at least one wave antinode and at least one wave trough.

According to one embodiment, the blade element can be described or defined by one or more camber lines, each having at least one inflection point, and/or a locally constant or variable thickening, in particular wherein a tangent slope of a tangent representing a curvature of the camber line initially increases in the direction of flow and decreases again after the inflection point to produce the wavy blade curvature. As a result, the wavy blade curvature in the direction of the camber line can be produced.

In the present case, the camber line of a blade element is in particular to be understood to be the center line of a profile of the blade element in an outer surface of a cylinder coaxial to the axis of rotation of the pump rotor, which connects the centers of all circles that fit into the profile, i.e. the curve that has the same (transverse) distance to the top and bottom of the profile throughout.

According to a further embodiment, an outer surface of the pump housing can have at least one outlet opening for lateral discharge of the conveying medium. In the installed state of the pump rotor, the blade section can be at least partially opposite to the outlet opening. This ensures efficient and gentle radial or diagonal discharge of the conveying medium.

The inflection point can be located in the region of the outlet opening, for example, in particular in the region of an edge of the outlet opening which is upstream when the axial flow pump is in operation, i.e. for example at the start of the outlet opening. This can further increase the efficiency of the axial flow pump.

According to a further embodiment, the blade element can be wound helically around the hub starting from a beginning of the pump rotor, wherein the tangent slope increases starting from the beginning of the pump rotor and decreases again after the inflection point. Such an embodiment increases the efficiency of the axial flow pump particularly well with reduced blood damage.

According to a further embodiment, the wavy blade curvature can vary in the direction of a radial extension of the blade element. A radial extension can be understood to be an extension in radial direction starting from the axis of rotation. As a result, the blade curvature can be optimally adapted to different boundary conditions in multiple directions.

According to a further embodiment, the wavy blade curvature can increase with increasing distance from the axis of rotation. This allows the greatest possible efficiency of the axial flow pump to be achieved.

It is also advantageous if the hub has a diameter that increases in the direction of flow. This supports the gentle discharge of the conveying medium in radial or diagonal direction to the axis of rotation.

The approach presented here also creates a method for producing an axial flow pump for a cardiac support system, whereby the method comprises the following steps:
forming a pump housing for arrangement in a blood vessel and
a pump rotor which consists of a hub and at least one blade element which is wound at least in sections helically around the hub for conveying a conveying medium, wherein the blade element comprises at least one blade section having a wavy blade curvature; and
   arranging the pump rotor in the pump housing, wherein the pump rotor is mounted such that it can rotate about an axis of rotation in order to convey the conveying medium in the direction of the axis of rotation.

This method can be implemented in software or hardware, for example, or in a mixed form of software and hardware, for example in a control device.

The approach presented here further creates a device that is configured to carry out, control and/or implement the steps of a variant of a method presented here in corresponding devices. The underlying object the invention can also be achieved quickly and efficiently with this design variant of the invention in the form of a device.

For this purpose, the device can comprise at least one computing unit for processing signals or data, at least one memory unit for storing signals or data, at least one interface to a sensor or an actuator for inputting sensor signals from the sensor or for outputting data or control signals to the actuator and/or at least one communication interface for inputting or outputting data that is embedded in a communication protocol. The computing unit can, for example, be a signal processor, a microcontroller, or the like, whereby the memory unit can be a flash memory, an EPROM, or a magnetic memory unit. The communication interface can be configured to input or output data in a wireless and/or wired manner, whereby a communication interface that can input or output wired data can, for example, input or output said data electrically or optically from or to a corresponding data transmission line.

A computer program product or computer program having a program code which can be stored on a machine-readable carrier or storage medium such as a semiconductor memory, a hard drive memory or optical memory and can be used to execute, implement and/or control the steps of the method according to one of the embodiments described above is also advantageous, in particular if the program product or program is executed on a computer or a device.

Advantageous design examples of the invention are schematically shown in the drawings and explained in more detail in the following description.

### BRIEF DESCRIPTION OF THE DRAWINGS

The figures show:

FIG. 1 a cardiac support system having an axial flow pump according to one design example;

FIG. 2 the axial flow pump of the cardiac support system of FIG. 1 having blade elements and having a hub in a side view;

FIG. 3 an unwinding of a blade element of the axial flow pump of FIGS. 1 and 2;

FIG. 4 a perspective partial view of the axial flow pump of FIGS. 1 and 2 with a blade element having a blade section with a wavy blade curvature and, for comparison, the geometry of a conventional blade element for an axial flow pump that does not have a wavy curved section;

FIG. 5 a view of the blade element of the axial flow pump shown in FIG. 1 and FIG. 2 with different blade heights relative to the surface of the hub;

FIG. 6 the profile of the blade angle β formed between the circumferential direction and a camber line of the blade element along a normalized meridional coordinate m/mMax;

FIG. 7 a flow diagram of a method for producing an axial flow pump according to one design example; and

FIG. 8 a schematic illustration of a device for carrying out and/or controlling the method of FIG. 7.

### DETAILED DESCRIPTION

In the following description of favorable design examples of the present invention, the same or similar reference signs are used for the elements shown in the various figures, which have a similar effect, whereby a repeated description of these elements is omitted.

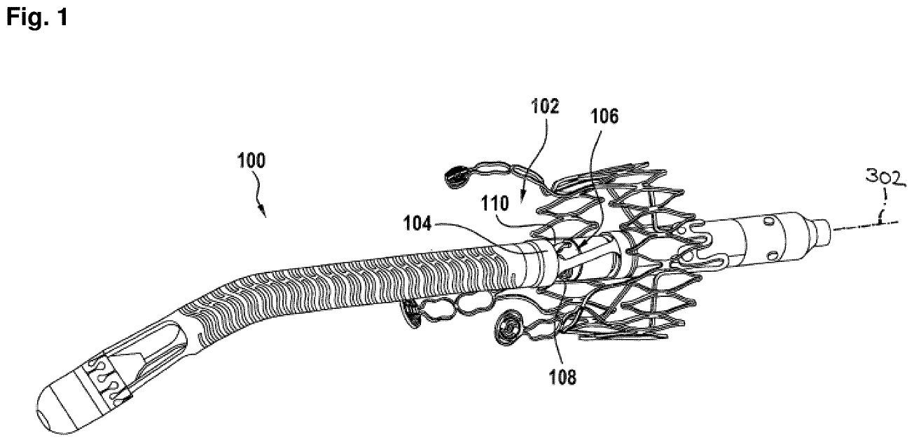

FIG. 1 shows a schematic illustration of a cardiac support system 100 having an axial flow pump 102 according to one design example. The cardiac support system 100 has a tube or hose-shaped configuration. The cardiac support system 100 is realized as an axial centrifugal pump system that can be placed into a blood vessel such as the aorta or a ventricle in a minimally invasive manner by means of a catheter. The axial flow pump 102 has an axis of rotation 302 and includes a pump housing 104 which is realized as a tube-shaped section of the cardiac support system 100 that surrounds the axis of rotation 302 and comprises, for example three, opposite lateral outlet openings 106 for laterally discharging a conveying medium, in this case blood. According to alternative design examples, the pump housing 104 comprises only one, two, three or even more than three outlet openings 106 distributed in circumferential direction. A pump rotor 108, also referred to as an impeller, is rotatably mounted in the pump housing 104 and, in the assembled state of the axial flow pump 102 shown in FIG. 1, said pump rotor is opposite to the outlet openings 106 in some sections. The pump rotor 108 serves to axially draw in and radially or diagonally expel the blood via the outlet openings 106.

To ensure the most efficient and gentle transport of the blood possible, the pump rotor 108 comprises at least one helically wound blade element 110. The camber line of the blade element 110 comprises an inflection point in the region of the upstream start of the outlet openings 106.

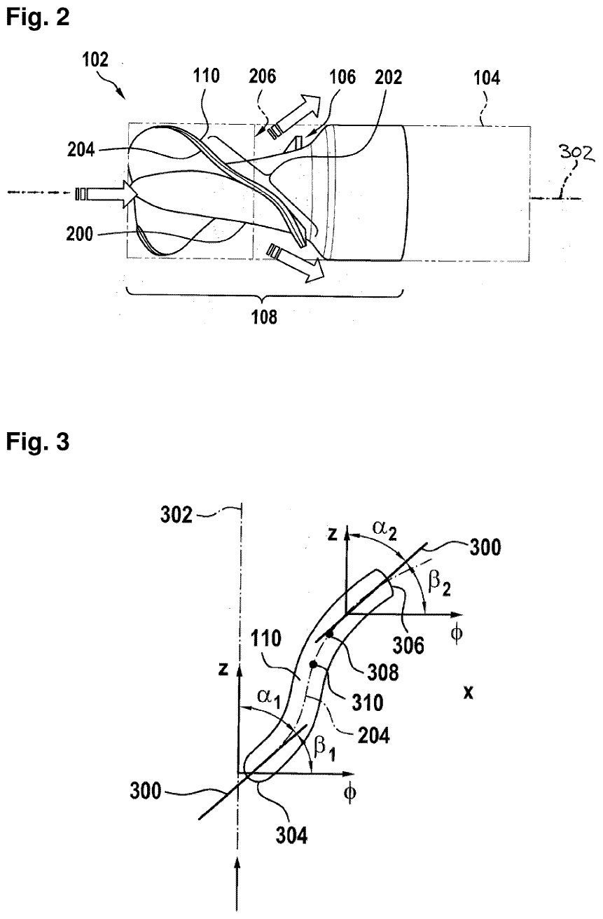

FIG. 2 shows a schematic illustration of the axial flow pump 102 of FIG. 1 in a side view. The blade element 110, which is wound helically around a hub 200 of the axial flow pump 102, can be seen. The hub 200 forms an inner core of the pump rotor 108. A flow direction of the conveying medium is schematically indicated with three arrows. The conveying medium is drawn in through a front-side inlet opening in the pump housing 104, which is upstream of the pump rotor 108 and functions as an intake opening.

According to this design example, the blade element 110 extends from an upstream end of the pump rotor 108 over an entire length or at least a majority of the hub 200. The hub 200 has a diameter that increases in the direction of flow, which results in a configuration of the hub 200 that becomes thicker in the direction of flow. This facilitates radial or diagonal discharge of the conveying medium.

The blade element 110 comprises a blade section 202 having a wavy blade curvature, which is defined by a multiple curvature of a camber line 204 of the blade element 110. A wavy blade curvature is to be understood here to be a change in the curvature of the blade section 202 associated with at least one change of sign.

As can be seen from FIG. 2, in the installed state of the pump rotor 108 at least one partial section of the blade section 202 is opposite to the outlet openings 106.

According to this design example, the blade section 202 is located at least partially in the region of a flow-facing edge 206 of the outlet opening 106. The blade section 202 represents a transition between a convex and a concave curvature.

As an example, the pump rotor 108 according to FIG. 2 comprises two blade elements 110, which are wound in the same direction around the hub 200 and each comprise the blade section 202. According to an alternative design example, the pump rotor 108 is realized with more than two such blade elements 110.

FIG. 3 shows a schematic illustration of the unwinding of a camber line 204 of a blade element 110 of FIGS. 1 and 2. Two pairs of blade angles α_{1d}, β₁ and α₂, β₂ are drawn in as an example, each of which represents a tangent slope of a tangent 300 representing the curvature of the camber line 204. Each tangent 300 is drawn into a cylindrical coordinate system with a z-axis parallel to an axis of rotation 302 of the pump rotor and a Φ-axis perpendicular to the z-axis. The Φ-axis represents a circumferential direction of the pump rotor.

As can be seen from FIG. 3, the tangent slope initially increases in flow direction, indicated by a vertical arrow, and then decreases again. According to this design example, the tangent slope initially increases continuously from a blade leading edge 304 to a blade trailing edge 306 of the blade element 110 and, upon reaching the inflection point 310 of the camber line 204, decreases again. A point 308 marks a position of a flow discharge via the outlet openings of the pump housing, more precisely a start of the flow discharge in axial direction. The objective here is to ensure that the inflection point 310 and the point 308 of the start of the flow discharge are in close proximity.

As already described, according to one design example, the pump rotor is realized with at least two blade elements 110. The conveying medium is delivered axially to or is drawn in by the pump rotor and expelled radially and diagonally through one or more outlet openings in the pump housing. The blade elements 110 are configured such that the angle between the tangent 300 formed with a blade surface or the camber line 204 and the axis of rotation 302 or the z-axis changes in axial direction. The angle β between the circumferential direction or the Φ-axis and the blade surface or the camber line 204 changes to the opposite extent. The angle β changes such that, at least in the region of the largest diameter of the pump rotor, i.e. in a section in the region of the blade tips of the blade elements 110, from the start of the pump rotor, i.e. from the blade leading edge 304, it increases in flow direction. The angle β in particular assumes its greatest value in the region of the start of the flow discharge 308 or in close proximity thereof, at least in the region of the largest diameter of the pump rotor, i.e. in a section in the region of the blade tips of the blade elements 110.

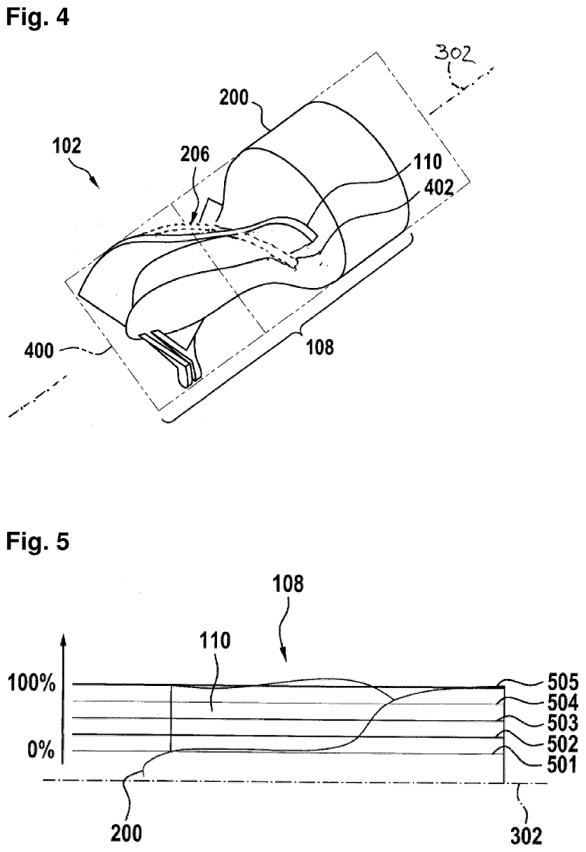

FIG. 4 shows a schematic illustration of the axial flow pump 102 of FIGS. 1 and 2 in perspective view. A region of the pump rotor 108 enclosed by the pump housing 104, in which the pump housing 104 has no outlet opening, is marked with a rectangle 400. The wavy blade curvature of the blade element 110, which is extended in a region enclosed by the pump housing 104 in which said housing has one outlet opening 106, is clearly visible. A conventional blade element 402 without a wavy blade curvature is drawn in next to the blade element 110 for comparison.

FIG. 5 is a view of the blade element of the axial flow pump shown in FIG. 1 and FIG. 2 with different blade heights relative to the surface of the hub of the pump rotor 108. Five horizontal lines representing different blade heights of the blade element 110 relative to a surface of the hub 200 are shown. A first line 501 represents 0 percent, a second line 502 represents 25 percent, a third line 503 represents 50 percent, a fourth line 504 represents 75 percent, and a fifth line 505 represents 100 percent of a maximum blade height.

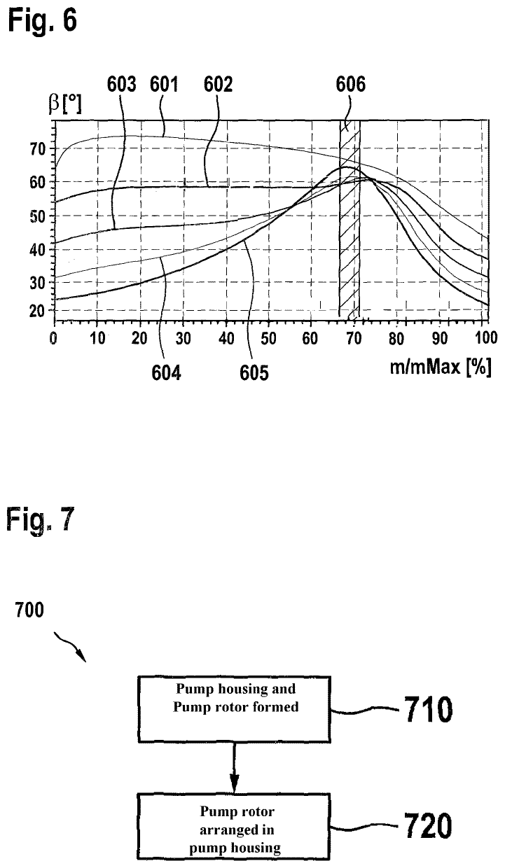

FIG. 6 shows a diagram to illustrate a progression of the blade angle β of FIG. 3 along a normalized meridional coordinate m/mMax. With the aid of five curves, the progression of the blade angle β is shown for different blade sections as a function of the blade height of the blade element, i.e. of a radial distance of the camber line thereof from the hub. Analogous to FIG. 5, a first curve 601 corresponds to a section of the blade element along the first line 501 at the height of the hub at 0 percent blade height, a second curve 602 corresponds to a section of the blade element along the second line 502 at 25 percent blade height, a third curve 603 corresponds to a section of the blade element along the third line 503 at 50 percent blade height, a fourth curve 604 corresponds to a section of the blade element along the fourth line 504 at 75 percent blade height and a fifth curve 605 corresponds to a section of the blade element along the fifth line 505 at 100 percent blade height, i.e. on the blade tips at the maximum impeller diameter. The start of the flow discharge at the outlet openings of the pump housing is marked with a hatched region 606.

On the one hand, the diagram shows that the blade angle β, and thus the curvature of the camber line, has different progressions in flow direction depending on the radial distance from the hub. On the other hand, it can be seen that the blade angle β in the considered sections initially increases in the direction of flow and decreases again after a high point, which in this case represents a respective maximum of the curves or the blade angle β. The position of the high point along the meridional coordinate varies depending on the radial distance of the camber line from the hub.

According to FIG. 6, the reversal point for the 25 to 100 percent blade heights is either in or just before or just after the hatched region 606, the distance of which from the inlet opening of the pump housing 104 viewed in the direction of the axis of rotation 302 corresponds to the distance of the edge of the outlet opening 106 of the pump housing 104 facing the inlet opening of the pump housing 104. Therefore, with respect to a similar coordinate origin located on the axis of rotation 302, the side of the outlet opening 106 facing the inlet opening of the pump housing 104 and the region of the pump rotor 108 corresponding to the hatched area 606 have axial coordinates that correspond to one another, i.e. which are equal to one another or deviate from one another only slightly.

In contrast, the reversal point for the 0 percent blade height is clearly located in front of the hatched region 606, here in a region of the meridional coordinate between 10 and 20 percent.

According to one design example, the pump rotor has the high point in the blade angle β in the region of the start of the flow discharge via the outlet openings or even in close proximity to the flow discharge, but at least in the outer area at the blade tips. In this region there is therefore a change in the curvature of the camber line from concave to convex. This design of the blade element allows the swirl in the flow to be reduced, which enables efficient operation and, associated with this, gentle fluid conveyance with reduced damage.

FIG. 7 shows a flow diagram of a method 700 for producing an axial flow pump according to one design example, for example the axial flow pump described above with reference to FIGS. 1 to 6. In a step 710, the pump housing and the pump rotor having the hub and the blade element wound helically around the hub are formed. The blade element is configured at least in sections with a wavy blade curvature. In a further step 720, the pump rotor is arranged in the pump housing. The pump rotor is mounted such that it can rotate about an axis of rotation in order to convey the conveying medium in the direction of the axis of rotation.

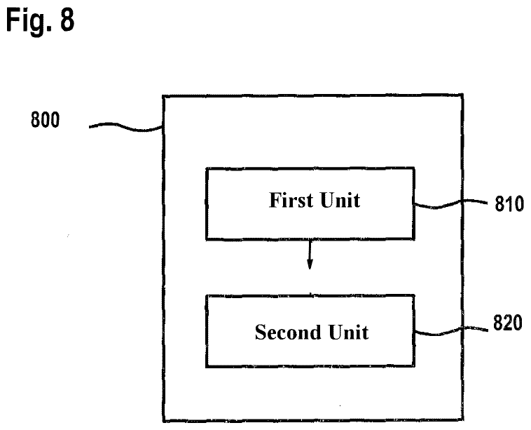

FIG. 8 shows a schematic illustration of a device 800 for carrying out and/or controlling the method 700 of FIG. 7. The device 800 comprises a first unit 810 for forming the pump housing and the pump rotor and a second unit 820 for arranging the pump rotor in the pump housing.

If a design example includes an “and/or” conjunction between a first feature and a second feature, this should be read to mean that the design example according to one embodiment comprises both the first feature and the second feature and, according to another embodiment, comprises either only the first feature or only the second feature.

In summary, the following features of the invention should in particular be noted: An axial flow pump 102 for a cardiac support system comprises a pump housing 104 for arrangement in a blood vessel and a pump rotor 108 which is or can be mounted in the pump housing 104 such that it can rotate about an axis of rotation and consists of a hub 200 and at least one blade element 110 which is wound at least in sections helically around the hub 200 for conveying a conveying medium in the direction of the axis of rotation 302. To increase the pump efficiency, the blade element 110 comprises at least one blade section 202 having a wavy blade curvature.

The invention in particular relates to the aspects specified in the following clauses:
1. Axial flow pump (102) for a cardiac support system (100), wherein the axial flow pump (102) has the following features:
   a pump housing (104) for arrangement in a blood vessel; and
   a pump rotor (108) which is or can be mounted in the pump housing (104) such that it can rotate about an axis of rotation (302) and consists of a hub (200) and at least one blade element (110) which is wound at least in sections helically around the hub (200) for conveying a conveying medium in the direction of the axis of rotation (302), wherein the blade element (110) comprises at least one blade section (202) having a wavy blade curvature.
2. Axial flow pump (102) according to Clause 1, in which the blade element (110) is defined by one or more camber lines (204), each having at least one inflection point, and/or a locally variable thickening, in particular wherein a tangent slope of a tangent (300) representing a curvature of the camber line (204) initially increases in the direction of flow and decreases again after the inflection point to produce the wavy blade curvature.
3. Axial flow pump (102) according to any one of the preceding clauses, in which an outer surface of the pump housing (104) comprises at least one outlet opening (106) for lateral discharge of the conveying medium, wherein in the installed state of the pump rotor (108), the blade section (202) is at least partially opposite to the outlet opening (106).
4. Axial flow pump (102) according to Clause 2 and 3, in which the inflection point of at least one camber line is located in the region of the outlet opening (106), in particular in the region of a flow-facing or upstream edge (206) of the outlet opening (106).
5. Axial flow pump (102) according any one of Clauses 2 to 4, in which the blade element (110) is wound helically around the hub (200) starting from a beginning (304) of the pump rotor (108), wherein the tangent slope increases starting from the beginning (304) of the pump rotor (108) and decreases again after the inflection point.
6. Axial flow pump (102) according to any one of the preceding clauses, in which the wavy blade curvature varies in the direction of a radial extension of the blade section (202).
7. Axial flow pump (102) according to any one of the preceding clauses, in which the wavy blade curvature increases with increasing distance from the axis of rotation (302).
8. Axial flow pump (102) according to any one of the preceding clauses, in which the hub (200) has a diameter which increases in the direction of flow.
9. Method (700) for producing an axial flow pump (102) for a cardiac support system (100), wherein the method (700) comprises the following steps:
   forming (710) a pump housing (104) for arrangement in a blood vessel and a pump rotor (108) which consists of a hub (200) and at least one blade element (110) which is wound at least in sections helically around the hub (200) for conveying a conveying medium, wherein the blade element (110) comprises at least one blade section (202) having a wavy blade curvature, and
   arranging (720) the pump rotor (108) in the pump housing (104), wherein the pump rotor (108) is mounted such that it can rotate about an axis of rotation (302) in order to convey the conveying medium in the direction of the axis of rotation (302).
10. Device (800) comprising units (810, 820), which are configured to carry out and/or control the method (700) according to claim 9.
11. Computer program, which is configured to execute and/or control the method (700) according to Clause 9.
12. Machine-readable storage medium, on which the computer program according to Clause 11 is stored.

What is claimed is:

1. A cardiac support system for insertion through a catheter into a blood vessel, comprising:
an axial flow pump comprising:
a tubular housing;
an intake portion disposed in a distal portion of the tubular housing;
at least one peripheral outlet opening disposed in a proximal portion of the tubular housing; and
a pump rotor disposed within the tubular housing, the pump rotor comprising a hub and at least one blade element, the at least one blade element being wound at least partially helically around the hub and configured to draw blood into the intake portion toward the proximal portion of the tubular housing;
wherein at least a portion of the at least one blade element is disposed proximal to a distal edge of the at least one peripheral outlet opening; and
wherein the at least one blade element transitions from having a convex curvature to having a concave curvature in a region of the at least one peripheral outlet opening.

2. The cardiac support system according to claim 1, wherein the at least one blade element transitions from having a convex curvature to having a concave curvature proximal to a distal edge of the at least one peripheral outlet opening.

3. The cardiac support system according to claim 1, wherein the hub faces a distal end of the cardiac support system.

4. The cardiac support system according to claim 1, wherein a thickness of the hub increases from an upstream end of the hub to a downstream end of the hub.

5. The cardiac support system according to claim 1, wherein the at least one blade element has a profile with camber lines, and wherein a curvature of each of the camber lines increases extending proximally from the intake portion toward the at least one peripheral outlet opening to an inflection point wherein the curvature of each of the camber lines decreases after the inflection point.

6. The cardiac support system according to claim 1, wherein the at least one blade element comprises at least one blade section having a wavy blade curvature.

7. The cardiac support system according to claim 6, wherein the at least one blade section having the wavy blade curvature is configured in a section of the pump rotor at least partially located in a housing section of the tubular housing open to the at least one peripheral outlet opening.

8. The cardiac support system according to claim 6, wherein the wavy blade curvature varies in a direction of a radial extension of the at least one blade section.

9. The cardiac support system according to claim 6, wherein the wavy blade curvature increases with in a direction of a radial extension of the at least one blade section.

10. The cardiac support system according to claim 6, wherein the at least one blade element has a thickness that changes in a direction of a radial extension in the at least one blade section having the wavy blade curvature.

11. The cardiac support system according to claim 10, wherein an inflection point of each of the camber lines of a radially outermost portion of the at least one blade element is proximal to a distal edge of the at least one peripheral outlet opening.

12. A cardiac support system for insertion through a catheter into a blood vessel, comprising:
an axial flow pump comprising:
at least one peripheral outlet opening; and
a pump rotor positioned at least partially distal from the at least one peripheral outlet opening, the pump rotor comprising a hub and at least one blade element, the at least one blade element being wound at least partially helically around the hub and configured to draw blood into an intake portion toward the at least one peripheral outlet opening;
wherein at least a portion of the at least one blade element is disposed proximal to a distal edge of the at least one peripheral outlet opening; and
wherein the at least one blade element transitions from having a convex curvature to having a concave curvature in a region of the at least one peripheral outlet opening.

13. The cardiac support system according to claim 12, wherein the hub faces a distal end of the cardiac support system.

14. The cardiac support system according to claim 12, wherein the at least one blade element comprises at least one blade section having a wavy blade curvature.

15. The cardiac support system according to claim 14, wherein the wavy blade curvature varies in a direction of a radial extension of the at least one blade section.

16. The cardiac support system according to claim 14, wherein the wavy blade curvature increases in a direction of a radial extension of the at least one blade section.

17. The cardiac support system according to claim 14, wherein the at least one blade element has a thickness that changes in the at least one blade section having the wavy blade curvature.

18. The cardiac support system according to claim 14, wherein the at least one blade section having the wavy blade curvature is configured in a section of the pump rotor at least partially located in a section of the axial flow pump open to the at least one peripheral outlet opening.

19. The cardiac support system according to claim 18, wherein the hub has a diameter that increases starting from a distal end of the pump rotor towards the at least one peripheral outlet opening.
